(19) United States
(12) Patent Application Publication
(10) Pub. No.: US 2013/0245399 A1
(43) Pub. Date: Sep. 19, 2013

(54) APPARATUS AND METHOD FOR MEASURING SKIN MOISTURE

(71) Applicants: Jea-Boong CHOI, Yongin-si (KR); Sang Mo YANG, Seoul (KR); Jung Min LEE, Hanam-si (KR); Sang Hun SUL, Seoul (KR); Kyung Shik OH, Suwon-si (KR)

(72) Inventors: Jea-Boong CHOI, Yongin-si (KR); Sang Mo YANG, Seoul (KR); Jung Min LEE, Hanam-si (KR); Sang Hun SUL, Seoul (KR); Kyung Shik OH, Suwon-si (KR)

(73) Assignee: RESEARCH & BUSINESS FOUNDATION SUNGKYUNKWAN UNIVERSITY, Suwon-si (KR)

(21) Appl. No.: 13/834,156

(22) Filed: Mar. 15, 2013

(30) Foreign Application Priority Data
Mar. 15, 2012 (KR) 10-2012-0026808

(51) Int. Cl. A61B 5/053; A61B 5/00

(52) U.S. Cl. CPC A61B 5/0537; A61B 5/443; A61B 5/6817; A61B 5/6898; A61B 5/72; A61B 5/742
USPC 600/306

(57) ABSTRACT

A skin moisture measuring apparatus that measures moisture in a testee's skin includes an earphone plug that is connected to an earphone port of a smart device and connects the skin moisture measuring apparatus to the smart device; an oscillation signal generation unit that generates an oscillation signal; and a sensor unit that includes two or more contact portions exposed to the outside to be in direct with the testee's skin, modifies the oscillation signal depending on an electrical resistance corresponding to a moisture content in the testee's skin and formed between the two or more contact portions, and generates a measurement signal. In the skin moisture measuring apparatus, the sensor unit transmits the measurement signal to the smart device through the earphone plug.

### CROSS-REFERENCE TO RELATED APPLICATION

This application claims the benefit of Korean Patent Application No. 2012-0026808 filed on Mar. 15, 2012, the entire disclosures of which are incorporated herein by reference.

### FIELD OF THE INVENTION

The present disclosure relates to an apparatus and a method for measuring moisture of a testee's skin, and particularly relates to a skin moisture measuring apparatus capable of providing measurement information of moisture of a testee's skin by using smart devices and a skin moisture measuring method using the same.

### BACKGROUND OF THE INVENTION

A smart device is a mobile terminal device that is not limited in function and can be modified or extended in function by using an application program (generally, referred to as “application” or “app”). By way of example, the smart device may include a smart phone, a smart TV, a smart key, a smart card, and a tablet computer (or a smart pad).

By way of example, a smart phone combines a mobile phone (cellular phone) and a personal digital assistant (PDA) and is designed for scheduling, sending and receiving faxes, and data communication such as internet communication in addition to functions of a mobile phone. Particularly, unlike a conventional mobile phone sold and used as a complete product, the smart phone has functions which can be extended in various ways by additionally installing, executing and deleting various applications as well as functions of a mobile phone.

Typically, such a smart device performs a data processing with respect to external devices through wired/wireless data communication such as a USB and Bluetooth. In order to do so, the smart device requires a communication interface port for connection with the external devices and needs to use a complicated communication protocol. Further, the external devices require their own power supply sources.

Therefore, there is a need to connect a smart device with various external devices, perform a data processing, and supply power to the external devices in a more simple way.

In this regard, Korean Patent No. 10-0819270 (entitled “Earmicrophone apparatus for supplying power to a mobile terminal and the mobile terminal”) describes mobile terminal that includes a first conventional plug connected to an earset device and a second plug for supplying power to the earset device.

Meanwhile, skin is the soft outer covering of vertebrates and has functions such as protection, thermoregulation, excretion, respiration, and the like. It has been known that such functions of the skin are influenced by a skin condition, particularly a skin moisture content.

A lot of devices for measuring skin moisture have been disclosed.

By way of example, Korean Patent No. 10-0938403 (entitled “Device for measuring moisture on skin”) describes a skin moisture measuring apparatus that calculates a primary skin moisture content through skin moisture measurement and moisture measurement by using bioimpedance and then calculating a final skin moisture content by amending the primary skin moisture content with variables such as a temperature and humidity of the external environment.

Further, Korean Patent No. 10-0979886 (entitled “Sensor with multi skin checking means”) describes a skin condition measuring sensor capable of measuring skin moisture and skin elasticity at a time.

However, a conventional skin moisture measuring apparatus uses a personal computer or a separate device for measurement value analysis and input/output interface with a user, and, thus, the user requires a personal computer or a separate device including a program related to skin moisture measurement and needs to know how to use the device. Therefore, the conventional skin moisture measuring apparatus offers low transportability and convenience.

### BRIEF SUMMARY OF THE INVENTION

In view of the foregoing, illustrative embodiments of the present disclosure provide a skin moisture measuring method and a skin moisture measuring apparatus with high convenience and transportability that is easily connected to a smart device via an earphone port of the smart device and offers a measurement result of a moisture content in a testee's skin through the smart device.

In accordance with one aspect of an illustrative embodiment, there is provided a skin moisture measuring apparatus that measures moisture in a testee's skin. The skin moisture measuring apparatus includes an earphone plug that is connected to an earphone port of a smart device and connects the skin moisture measuring apparatus to the smart device; an oscillation signal generation unit that generates an oscillation signal; and a sensor unit that includes two or more contact portions exposed to the outside to be in direct with the testee's skin, modifies the oscillation signal depending on an electrical resistance corresponding to a moisture content in the testee's skin and formed between the two or more contact portions, and generates a measurement signal. In the skin moisture measuring apparatus, the sensor unit transmits the measurement signal to the smart device through the earphone plug.

If the smart device receives the measurement signal, the smart device may draw skin moisture information corresponding to the received measurement signal and display the skin moisture information.

Further, in accordance with another aspect of the illustrative embodiment, there is provided a skin moisture measuring method that measures moisture in a testee's skin by using a skin moisture measuring apparatus to be connected to a smart device through an earphone port of the smart device. The skin moisture measuring method includes generating an oscillation signal based on power supplied to the skin moisture measuring apparatus; and generating a measurement signal by modifying the oscillation signal depending on a moisture content in the testee's skin.

In accordance with the present disclosure, the skin moisture measuring apparatus can be easily connected to the smart device via the earphone port of the smart device. Further, the skin moisture measuring apparatus connected to the smart device measures the moisture content contained in the testee's skin and displays the measurement result through the smart device. Therefore, the skin moisture measuring apparatus in accordance with the present disclosure can offer improved transportability and convenience as compared with a case where skin moisture measurement is carried out by a personal computer or a separate device with low mobility.

Herein, the skin moisture measuring apparatus is supplied with power from the smart device and does not require an additional power supply source. Therefore, a size and a weight of the skin moisture measuring apparatus can be reduced, and, thus, transportability can be further improved.

### BRIEF DESCRIPTION OF THE DRAWINGS

Non-limiting and non-exhaustive embodiments will be described in conjunction with the accompanying drawings. Understanding that these drawings depict only several embodiments in accordance with the disclosure and are, therefore, not to be intended to limit its scope, the disclosure will be described with specificity and detail through use of the accompanying drawings, in which:

FIG. 1 is a block diagram illustrating a skin moisture measuring system in accordance with an illustrative embodiment;

FIG. 2 is a block diagram illustrating an input/output port of FIG. 1;

FIG. 3 a block diagram illustrating a smart device of FIG. 1;

FIG. 4 is a block diagram illustrating a skin moisture measuring apparatus of FIG. 1;

FIG. 5 provides an example of the skin moisture measuring apparatus of FIG. 4;

FIG. 6 provides an example of the skin moisture measuring system of FIG. 1;

FIG. 7 is a flow chart illustrating a skin moisture measuring method using a smart device in accordance with an illustrative embodiment;

FIG. 8 is a flow chart illustrating a step of executing a skin moisture measurement application of FIG. 7; and

FIG. 9 is a flow chart illustrating a process of transmitting a measurement signal of FIG. 8 by using a skin moisture measuring apparatus in accordance with an illustrative embodiment.

### DETAILED DESCRIPTION OF THE INVENTION

Hereinafter, examples of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, the term “comprises or includes” and/or “comprising or including” used in the document means that one or more other components, steps, operation and/or existence or addition of elements are not excluded in addition to the described components, steps, operation and/or elements unless context dictates otherwise.

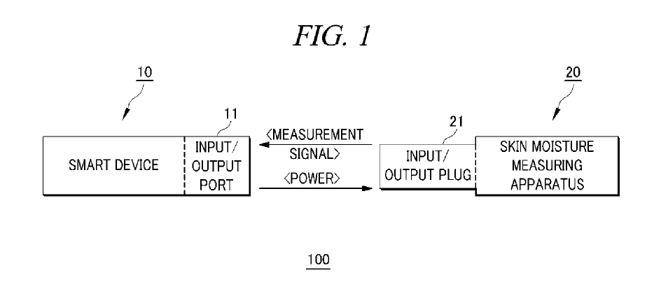

Hereinafter, a skin moisture measuring system in accordance with an illustrative embodiment will be explained in detail with reference to FIG. 1. FIG. 1 is a block diagram illustrating a skin moisture measuring system in accordance with an illustrative embodiment.

As depicted in FIG. 1, a skin moisture measuring system 100 in accordance with an illustrative embodiment includes a smart device 10 and a skin moisture measuring apparatus 20.

The smart device 10 is a mobile terminal that has preset functions and can be modified or extended in function by additionally installing, executing, and deleting an application program.

The smart device 10 includes at least one input/output port 11 for performing a data processing and supplying power to external devices.

The input/output port 11 is connected to any one earphone plug of multiple external devices which can be connected to the smart device 10. Herein, the multiple external devices may include a data processing device, such as a printer, a keyboard, a joy stick, and a sensor, configured to be connected to the smart device 10 and perform data communication for executing various applications.

Typically, the smart device 10 includes an earphone port to be connected to a sound system including a speaker or an earphone that converts an electric signal to a sound for listening to music and movies, a microphone that converts a sound to an electric signal for recording, and an earset (earphone-microphone set) that combines an earphone with a microphone for hands-free phone call. Herein, the earphone port may be a socket of a cylindrical connector.

The skin moisture measuring apparatus 20 in accordance with an illustrative embodiment is connected to the smart device 10 via the earphone port among the multiple input/output ports 11 provided in the smart device 10. Thus, the smart device 10 does not require a separate input/output port for connection between the smart device 10 and the skin moisture measuring apparatus 20.

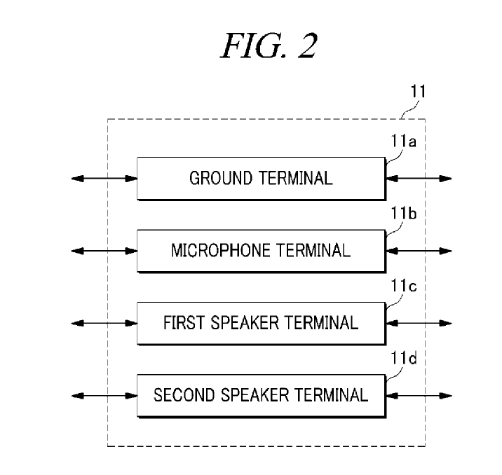

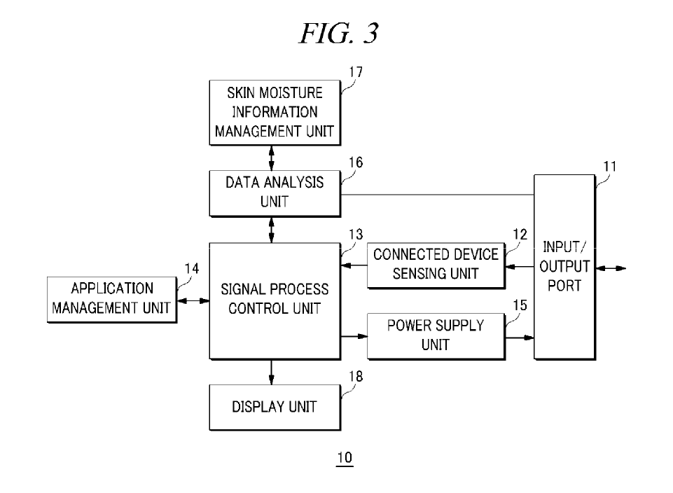

The smart device 10 and the input/output port 11 included therein will be explained in more detail with reference to FIGS. 2 and 3.

The skin moisture measuring apparatus 20 is connected to the smart device 10 via the input/output port 11 and driven by the smart device 10.

That is, when the smart device 10 is connected to the skin moisture measuring apparatus 20 via the input/output port 11, the smart device 10 supplies power to the skin moisture measuring apparatus 20. If the skin moisture measuring apparatus 20 is in contact with a testee's skin selected by a user while being supplied with power from the smart device 10, the skin moisture measuring apparatus 20 transmits a measurement signal corresponding to a moisture content in the testee's skin to the smart device 10. When the smart device 10 receives the measurement signal from the skin moisture measuring apparatus 20, the smart device 10 draws skin moisture information corresponding to the received measurement signal and displays the drawn skin moisture information for the user to see.

The skin moisture measuring apparatus 20 includes an input/output plug 21 connected to the input/output port 11 in order to be connected to the smart device 10. Herein, the input/output plug 21 may be an earphone plug matched with the earphone port 11. By way of example, the earphone plug 21 may be a cylindrical plug which can be connected to the earphone port 11.

Hereinafter, the smart deice 10 of FIG. 1 will be explained in more detail with reference to FIGS. 2 and 3. FIG. 2 is a block diagram illustrating an input/output port of FIG. 1 and FIG. 3 a block diagram illustrating a smart device of FIG. 1.

As depicted in FIG. 2, the input/output port 11 of the smart device 10 may be an earphone port including a ground terminal 11a, a microphone terminal 11b, and first and second speaker terminals 11c and 11d.

By way of example, if an earphone plug of an earset (not illustrated) among the multiple external devices is connected to the earphone port 11, a connection signal of the earset may be input to the microphone terminal 11b and an audio signal collected by a microphone (not illustrated) of the earset may be input to the microphone terminal 11b. The first and second speaker terminals 11c and 11d respectively output audio signals to both earphones (not illustrated) of the earset. In this case, an input terminal of the earphone plug of the earset is connected to the microphone terminal 11b and an output terminal thereof is connected to the first and second speaker terminals 11c and 11d.

If the earphone plug of the earset includes a ground terminal, the ground terminal of the earphone port of the smart device is connected to the ground terminal of the earphone plug of the earset, and, thus, the earset can be supplied with a ground voltage.

Further, at least one of the microphone terminal 11b and the first and second speaker terminals 11c and 11d may be used to supply power to the multiple external devices including the earset. That is, only one of the microphone terminal 11b and the first and second speaker terminals 11c and 11d may be used to supply power. Otherwise, all of the microphone terminal 11b and the first and second speaker terminals 11c and 11d may be used to supply power. The ground terminal 11a connected to the ground terminal of the earphone plug may be used to supply a ground voltage.

By way of example, if the earphone plug of the skin moisture measuring apparatus 20 is connected to the earphone port 11, power may be supplied to the skin moisture measuring apparatus 20 through the microphone terminal 11b of the earphone port. Otherwise, power may be supplied to the skin moisture measuring apparatus 20 through any one of the first and second speaker terminals 11c and 11d of the earphone port 11.

The microphone terminal 11b of the earphone port may receive a measurement signal from the skin moisture measuring apparatus 20. In this case, the measurement signal has a frequency selected from an audio sampling rate range of the smart device 10 in order to use the existing microphone terminal 11b. By way of example, the audio sampling rate range of the smart device 10 may be from about 0 Hz to about 22.05 kHz, particularly from about 30 Hz to about 22.05 kHz.

If the earphone plug 21 of the skin moisture measuring apparatus 20 includes a ground terminal, the ground terminal 11a of the earphone port is connected to the ground terminal of the earphone plug 21 so as to be used to supply a ground voltage to the skin moisture measuring apparatus 20.

As depicted in FIG. 3, the smart device 10 includes the input/output port 11, a connected device sensing unit 12, a signal process control unit 13, an application management unit 14, a power supply unit 15, a data analysis unit 16, skin moisture information management unit 17, and a display unit 18.

As described above, the input/output port 11 is an earphone port that is connected to an earphone plug of the external device and connects the smart device 10 to the external device.

If any one of input/output plugs of the multiple external devices is connected to the input/output port 11, the connected device sensing unit 12 receives a connection signal from the external device connected to the input/output port 11. Then, based on the received connection signal, the connected device sensing unit 12 senses the external device connected to the input/output port 11 and transmits information about the sensed external device to the signal process control unit 13.

Herein, the connection signal may be generated when the earphone plug of the external device is connected to the earphone port 11. By way of example, the connection signal may correspond to an initial power value of the external device or a voltage discharged to supply power to the external device.

Although not illustrated in detail in FIG. 3, the smart device may further include an external device management unit that contains a reference connection signal corresponding to various external devices. In this case, based on the reference connection signal corresponding to various external devices, the connected device sensing unit can find an external device to be matched with the received connection signal.

The signal process control unit 13 receives the information about the sensed external device from the connected device sensing unit 12 and executes a signal process application corresponding to the sensed external device.

Herein, the signal process application include an algorithm including supplying power to the external device, transmitting and receiving data to/from the external device, analyzing a signal received from the external device by a predetermined method, and outputting an analysis result.

In particular, if the connected device sensing unit 12 senses the skin moisture measuring apparatus 20 connected to the earphone port 11, the signal process control unit 13 executes a skin moisture measurement application.

Herein, the skin moisture measurement application includes an algorithm including supplying power to the skin moisture measuring apparatus 20, controlling the skin moisture measuring apparatus 20, and displaying a moisture content in a testee's skin based on a measurement signal received from the skin moisture measuring apparatus 20.

The application management unit 14 contains one or more signal process applications to be matched with various external devices which can be connected to the smart device as well as the skin moisture measurement application.

Then, the signal process control unit 13 searches at least one application corresponding to the sensed external device from various signal process applications contained in the application management unit 14 and is supplied with the at least one searched signal process application from the application management unit 14.

The power supply unit 15 supplies power to the connected external device through the earphone port 11 based on the signal process application executed by the signal process control unit 13. That is, the power supply unit 15 supplies power to the skin moisture measuring apparatus 20 through the earphone port 11 based on the skin moisture measurement application executed by the signal process control unit 13.

By way of example, the power supply unit 15 outputs a high-level signal continuously or periodically to any one of the microphone terminal 11b and the first and second speaker terminals 11c and 11d of the earphone port 11, so that the power supply unit 15 can supply power to the skin moisture measuring apparatus 20. If the power supply unit 15 outputs a high-level signal continuously or periodically to any one of the microphone terminal 11b and the first and second speaker terminals 11c and 11d of the earphone port 11, the skin moisture measuring apparatus 20 may include a rectifier circuit that converts a periodic high-level signal to power.

By way of example, the power supply unit 15 outputs a high-level signal to the microphone terminal 11b of the earphone port, so that the power supply unit 15 can supply power to the skin moisture measuring apparatus 20.

The data analysis unit 16 analyzes a data signal of the external device received through the input/output port in a predetermined method based on the signal process application executed by the signal process control unit 13 and allows an analysis result to be displayed on the display unit 18.

By way of example, the data analysis unit 16 receives the measurement signal from the skin moisture measuring apparatus 20 through the earphone port 11 based on the signal process application executed by the signal process control unit 13 and draws skin moisture information corresponding to the received measurement signal. Then, the skin moisture information is transmitted to the signal process control unit 13 so as to be displayed on the display unit 18.

The skin moisture information management unit 17 contains skin moisture information matched with each measurement signal.

The data analysis unit 16 searches skin moisture information matched with the received measurement signal from the skin moisture information contained in the skin moisture information management unit 17.

The display unit 18 displays a video signal or an audio signal based on the signal process application executed by the signal process control unit 13. By way of example, the display unit 18 may display the skin moisture information drawn by the data analysis unit 16 on a screen based on the skin moisture measurement application.

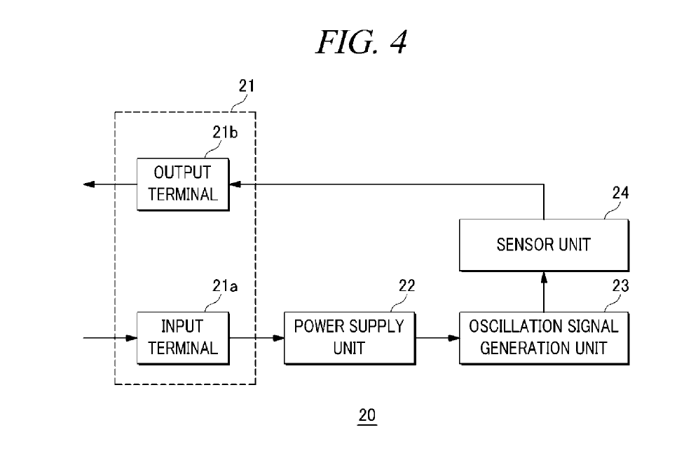

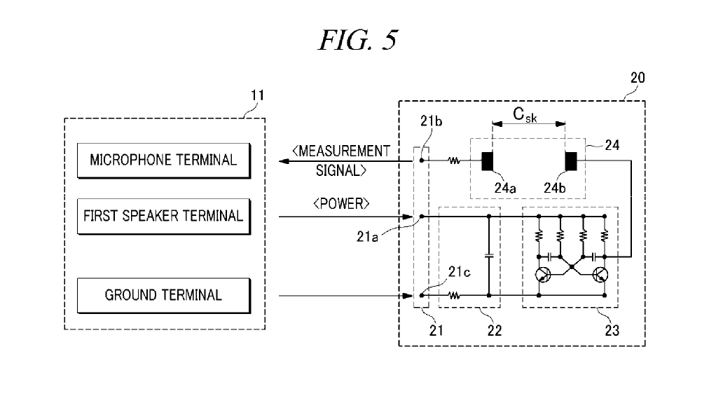

Hereinafter, the skin moisture measuring apparatus 20 of FIG. 1 will be explained in more detail with reference to FIGS. 4 and 5. FIG. 4 is a block diagram illustrating a skin moisture measuring apparatus of FIG. 1 and FIG. 5 provides an example of the skin moisture measuring apparatus of FIG. 4.

As depicted in FIG. 4, the skin moisture measuring apparatus 20 includes the earphone plug 21, a power supply unit 22, an oscillation signal generation unit 23, and a sensor unit 24.

The earphone plug 21 connected to the earphone port 11 of the smart device 10 connects the smart device 10 to the skin moisture measuring apparatus 20.

The power supply unit 22 applies a driving signal to the oscillation signal generation unit 23 based on power supplied from the outside of the skin moisture measuring apparatus 20. In particular, the power supply unit 22 may be supplied with power from the smart device connected to the earphone plug 21.

The sensor unit 24 includes two or more contact portions (not illustrated) exposed to the outside of a housing such that the sensor unit 24 can be in direct contact with the testee's skin. The sensor unit 24 generates a measurement signal corresponding to a moisture content in the testee's skin.

As depicted in FIG. 5, the earphone plug 21 includes an input terminal 21a connected to any one of the microphone terminal 11b and the first and second speaker terminals 11c and 11d of the earphone port to be supplied with power and an output terminal 21b connected to the microphone terminal 11b of the earphone port 11 to output a measurement signal.

Herein, the input terminal 21a is not only used to be supplied with power but also used to receive a control signal of the smart device 10 about the skin moisture measuring apparatus 20.

Further, the earphone plug 21 may further include a ground terminal 21c connected to the ground terminal 11a of the earphone port 11 to be supplied with a ground voltage.

The power supply unit 22 applies a driving signal to the oscillation signal generation unit 23 based on the power supplied from the smart device 10.

By way of example, the power supply unit 22 may be a capacitor connected between the input terminal 21a and the ground terminal 21c. The power supply unit 22 is charged with the power supplied from the smart device 10 through the input terminal 21a and discharged by the oscillation signal generation unit 23.

The oscillation signal generation unit 23 generates an oscillation signal by oscillating a driving signal applied from the power supply unit 22 to have a certain frequency. The oscillation signal generation unit 23 positively feeds back a part of an output signal amplified from the driving signal, so that the oscillation signal generation unit 23 may serve as an oscillation circuit that generates oscillation.

Herein, a frequency of the oscillation signal is selected from the audio sampling rate range of the smart device 10. By way of example, the audio sampling rate range of the smart device 10 may be from about 0 Hz to about 22.05 kHz, particularly from about 30 Hz to about 22.05 kHz in order to use the existing microphone terminal 11b for receiving an audio signal from the microphone when a measurement signal converted from the oscillation signal is input. If the generated oscillation signal has a frequency in the audio sampling rate range, the measurement signal also has a frequency in the audio sampling rate range, and, thus, it can be input to the smart device 10 through the existing microphone terminal 11b.

The sensor unit 24 includes two or more contact portions 24a and 24b which can be in contact with the testee's skin. Further, the sensor unit 24 modifies an oscillation signal depending on a moisture content in the testee's skin and generates a measurement signal.

Before the two or more contact portions 24a and 24b are in contact with the testee's skin, an electrical resistance sk between the contact portions 24a and 24b is maintained in a predetermined range by air or a separate dielectric member and does not correspond to a moisture content in the testee's skin. Therefore, a measurement signal is generated from an oscillation signal modified depending on the electrical resistance sk in the predetermined range between the contact portions 24a and 24b and can be analyzed as an initial value that does not correspond to the moisture content.

If the two or more contact portions 24a and 24b are in contact with the testee's skin, the electrical resistance sk corresponding to the moisture content in the testee's skin and formed between the contact portions 24a and 24b is modified so as to be out of the predetermined range.

That is, the electrical resistance sk between the contact portions 24a and 24b is modified in a range corresponding to the moisture content in the testee's skin and a variance in oscillation signal for generating a measurement signal is modified accordingly. By way of example, a variance in amplitude of the oscillation signal may be modified depending on the electrical resistance sk between the contact portions 24a and 24b.

Therefore, a measurement signal is generated from an oscillation signal modified depending on the electrical resistance sk corresponding to the moisture content in the testee's skin and formed between the contact portions 24a and 24b between the contact portions 24a and 24b and can be analyzed as a measurement value corresponding to the moisture content in the testee's skin. That is, a variance in oscillation signal is sensed from a difference between the measurement signal and the oscillation signal and the electrical resistance sk between the contact portions 24a and 24b is drawn, and based on the electrical resistance sk, the moisture content in the testee's skin is calculated.

The generated measurement signal is transmitted to the smart device 10 through the output terminal 21b of the earphone plug connected to the microphone terminal 11b of the earphone port.

The smart device 10 in accordance with the illustrative embodiment may include any mobile terminal in which a skin moisture measurement application can be installed.

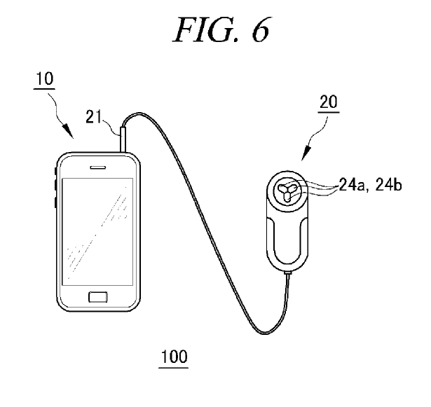

In particular, as depicted in FIG. 6, in the skin moisture measuring system 100, the smart device 10 may be a smart phone. Further, the skin moisture measuring apparatus 20 may be a small-sized device including the earphone plug to be connected to the earphone port 11 of the smart device 10 and the two or more contact portions 24a and 24b exposed to the outside of the housing in order to offer transportability and convenience.

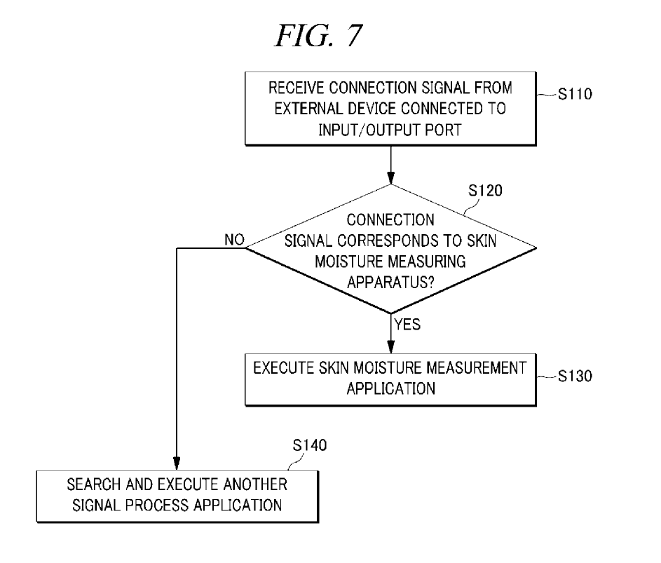

Hereinafter, a skin moisture measuring method in accordance with an illustrative embodiment will be explained with reference to FIGS. 7 to 9.

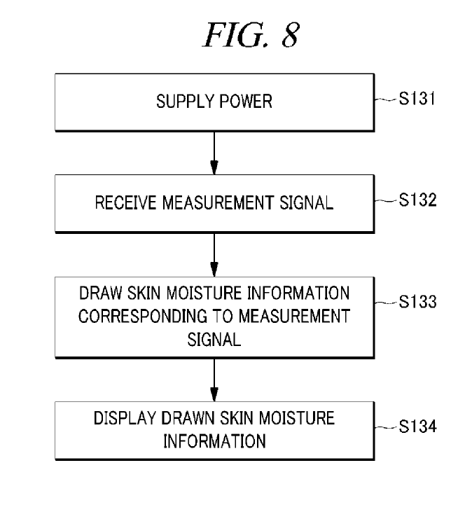

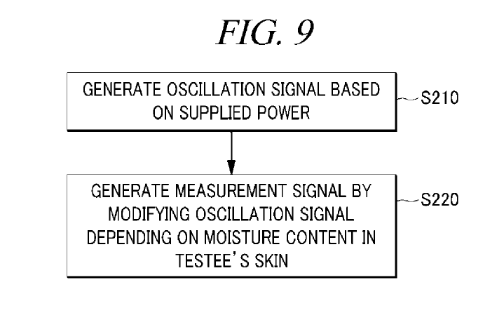

FIG. 7 is a flow chart illustrating a skin moisture measuring method using a smart device in accordance with an illustrative embodiment and FIG. 8 is a flow chart illustrating a step of executing a skin moisture measurement application of FIG. 7. FIG. 9 is a flow chart illustrating a process of transmitting a measurement signal of FIG. 8 by using a skin moisture measuring apparatus in accordance with an illustrative embodiment.

As depicted in FIG. 7, a skin moisture measuring method using the smart device 10 which can be connected to a skin moisture measuring apparatus through an input/output port includes a step (S110) of receiving a connection signal from an external device connected to the input/output port, a step (S120) of determining whether or not the received connection signal corresponds to the skin moisture measuring apparatus, and a step (S130) of executing a skin moisture measurement application if the received connection signal corresponds to the skin moisture measuring apparatus. If the received connection signal does not correspond to the skin moisture measuring apparatus, the method further includes a step (S140) of searching a signal process application corresponding to another external device and executing the searched signal process application.

As depicted in FIG. 8, the step (S130) of executing a skin moisture measurement application includes a step (S131) of supplying power to the skin moisture measuring apparatus, a step (S132) of receiving a measurement signal corresponding to a moisture content in a testee's skin from the skin moisture measuring apparatus, a step (S133) of drawing skin moisture information corresponding to the received measurement signal, and a step (S134) of displaying the skin moisture information.

As depicted in FIG. 9, a step of generating a measurement signal using the skin moisture measuring apparatus 20 includes a step (S210) of generating an oscillation signal based on supplied power and a step (S220) of modifying the oscillation signal depending on a moisture content in a testee's skin, generating a measurement signal, and transmitting the generated measurement signal. The oscillation signal can be generated based on power supplied from the smart device 10 (S210).

The above-described skin moisture measuring method will be explained in detail as follows.

The earphone port 11 of the smart device 10 is connected to the earphone plug 21 of the skin moisture measuring apparatus 20, so that the smart device 10 is connected to the skin moisture measuring apparatus 20. In this case, the skin moisture measuring apparatus 20 connected to the smart device 10 through the earphone port 11 transmits a connection signal to the smart device 10.

The smart device receives the connection signal (S110) and searches an external device corresponding to the received connection signal from multiple external devices which can be connected to the earphone port 11. At the same time, the smart device 10 determines whether or not the received connection signal corresponds to the skin moisture measuring apparatus 20 (S120).

If the received connection signal does not correspond to the skin moisture measuring apparatus 20, the smart device 10 executes a signal process application matched with the searched external device (S140).

That is, the smart device 10 re-searches another external device corresponding to the received connection signal from the other external devices except the skin moisture measuring apparatus 20 and executes the matched signal process application. Meanwhile, if the received connection signal corresponds to the skin moisture measuring apparatus 20, a skin moisture measurement application matched with the skin moisture measuring apparatus 20 is executed (S130).

The smart device 10 supplies power to the skin moisture measuring apparatus 20 connected to the earphone port 11 based on the executed skin moisture measurement application (S131). At the same time, the smart device 10 waits for a measurement signal of the skin moisture measuring apparatus 20.

The skin moisture measuring apparatus 20 is supplied with power from the smart device 10 and generates an oscillation signal based on the supplied power (S210).

Then, the skin moisture measuring apparatus 20 modifies the oscillation signal depending on an electrical resistance between two or more contact portions in contact with a testee's skin, generates a measurement signal, and transmits the generated measurement signal to the smart device 10 (S220).

Herein, the electrical resistance between two or more contact portions corresponds to a moisture content in the testee's skin, and, thus, a variance in oscillation signal, i.e. the measurement signal, represents a measurement value corresponding to the moisture content in the testee's skin.

If the smart device 10 receives the measurement signal from the skin moisture measuring apparatus 20 while executing the skin moisture measurement application, skin moisture information corresponding to the measurement signal is drawn (S133) and the skin moisture information is provided to a user (S134).

As described above, the skin moisture measuring system and the skin moisture measuring method using the system use the smart device 10 and the skin moisture measuring apparatus 20 to be connected to the earphone port 11 of the smart device 10, and, thus, transportability and convenience can be improved.

Herein, the skin moisture measuring apparatus is driven after being supplied with power from the smart device 10 through the earphone port 11. Thus, the skin moisture measuring apparatus does not require an additional battery or power supply unit. Therefore, a size and a weight of the skin moisture measuring apparatus 20 can be reduced, and, thus, transportability can be further improved.

The smart device 10 senses connection with the skin moisture measuring apparatus 20 based on a characteristic connection signal of the skin moisture measuring apparatus 20 and executes a signal process application matched with the connected skin moisture measuring apparatus 20. Thus, a skin moisture measurement application is executed just by connecting the skin moisture measuring apparatus 20 to the smart device 10. Therefore, inconvenience of the user can be reduced.

Further, if the user is skilled in using the smart device 10, the user can measure a moisture content in the testee's skin by using the skin moisture measuring apparatus without any training. Therefore, convenience can be further improved.

For reference, each of components illustrated in FIGS. 1 to 4 in accordance with the illustrative embodiment of the present disclosure may imply software or hardware such as a field programmable gate array (FPGA) or an application specific integrated circuit (ASIC), and they carry out a predetermined function.

However, the components are not limited to the software or the hardware, and each of the components may be stored in an addressable storage medium or may be configured to implement one or more processors.

Accordingly, the components may include, for example, software, object-oriented software, classes, tasks, processes, functions, attributes, procedures, sub-routines, segments of program codes, drivers, firmware, micro codes, circuits, data, database, data structures, tables, arrays, variables and the like.

The components and functions thereof can be combined with each other or can be divided.

The illustrative embodiments can be embodied in a storage medium including instruction codes executable by a computer or processor such as a program module executed by the computer or processor. A data structure in accordance with the illustrative embodiments can be stored in the storage medium executable by the computer or processor. A computer readable medium can be any usable medium which can be accessed by the computer and includes all volatile/non-volatile and removable/non-removable media. Further, the computer readable medium may include all computer storage and communication media. The computer storage medium includes all volatile/non-volatile and removable/non-removable media embodied by a certain method or technology for storing information such as computer readable instruction code, a data structure, a program module or other data. The communication medium typically includes the computer readable instruction code, the data structure, the program module, or other data of a modulated data signal such as a carrier wave, or other transmission mechanism, and includes information transmission mediums.

The system and method of the present disclosure has been explained in relation to a specific embodiment, but its components or a part or all of its operation can be embodied by using a computer system having general-purpose hardware architecture or desirably, a digital signal processing system to which an order of priority can be applied.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

What is claimed is:

1. A skin moisture measuring apparatus that measures moisture in a testee's skin, the skin moisture measuring apparatus comprising:
an earphone plug that is connected to an earphone port of a smart device and connects the skin moisture measuring apparatus to the smart device;
an oscillation signal generation unit that generates an oscillation signal; and
a sensor unit that includes two or more contact portions exposed to the outside to be in direct with the testee's skin, modifies the oscillation signal depending on an electrical resistance corresponding to a moisture content in the testee's skin and formed between the two or more contact portions, and generates a measurement signal,
wherein the sensor unit transmits the measurement signal to the smart device through the earphone plug.

2. The skin moisture measuring apparatus of claim 1,
wherein if the smart device receives the measurement signal, the smart device draws skin moisture information corresponding to the received measurement signal and displays the skin moisture information.

3. The skin moisture measuring apparatus of claim 1,
wherein a frequency of the oscillation signal is selected from an audio sampling rate range of the smart device.

4. The skin moisture measuring apparatus of claim 1, further comprising:
a power supply unit that is connected between the earphone plug and the oscillation signal generation unit and charged with power supplied from the outside and discharged by the oscillation signal generation unit.

5. The skin moisture measuring apparatus of claim 1,
wherein the oscillation signal generation unit generates the oscillation signal based on power supplied from the smart device.

6. The skin moisture measuring apparatus of claim 5,
wherein the smart device includes:
a connected device sensing unit that receives a connection signal from an external device connected to the earphone port when the earphone port is connected to any one of multiple external devices and senses the external device connected to the earphone port based on the received connection signal;
a signal process control unit that executes a skin moisture measurement application when the connected device sensing unit senses the skin moisture measuring apparatus among the multiple external devices;
a power supply unit that supplies power to the skin moisture measuring apparatus through the earphone port based on the executed skin moisture measurement application;
a data analysis unit that receives the measurement signal from the skin moisture measuring apparatus through the earphone port based on the executed skin moisture measurement application and draws the skin moisture information corresponding to the received measurement signal; and
a display unit that displays the skin moisture information based on the executed skin moisture measurement application.

7. The skin moisture measuring apparatus of claim 6,
wherein the smart device further includes:
an application management unit that contains at least one application corresponding to each of the multiple external devices which can be connected to the smart device; and
a skin moisture information management unit that contains skin moisture information matched with each measurement signal,
wherein the signal process control unit is supplied with the skin moisture measurement application from the application management unit when the connected device sensing unit senses the skin moisture measuring apparatus connected to the earphone port, and
the data analysis unit draws the skin moisture information corresponding to the received measurement signal among the skin moisture information matched with each measurement signal when the received measurement signal is modified in a predetermined error range.

8. The skin moisture measuring apparatus of claim 6,
wherein the earphone port of the smart device includes a ground terminal, a microphone terminal, and two speaker terminals, and
the smart device receives the measurement signal through the microphone terminal.

9. The skin moisture measuring apparatus of claim 8,
wherein the skin moisture measuring apparatus is supplied with power through any one of the microphone terminal and the two speaker terminals of the smart device, or
the skin moisture measuring apparatus is supplied with power through any one of the microphone terminal and the two speaker terminals of the smart device and through the ground terminal of the smart device.

10. A skin moisture measuring method that measures moisture in a testee's skin by using a skin moisture measuring apparatus to be connected to a smart device through an earphone port of the smart device, the skin moisture measuring method comprising:
generating an oscillation signal based on power supplied to the skin moisture measuring apparatus; and
generating a measurement signal by modifying the oscillation signal depending on a moisture content in the testee's skin.

11. The skin moisture measuring method of claim 10,
wherein the skin moisture measuring apparatus includes two or more contact portions exposed to the outside to be in direct with the testee's skin, and
the step of generating a measurement signal includes modifying the oscillation signal depending on an electrical resistance corresponding to the moisture content in the testee's skin and formed between the two or more contact portions.

12. The skin moisture measuring method of claim 10,
wherein in the step of generating an oscillation signal, the oscillation signal is generated based on power supplied from the smart device, and
the smart device executes a skin moisture measurement application based on a connection signal received from the skin moisture measuring apparatus connected to the earphone port,
supplies power to the skin moisture measuring apparatus based on the skin moisture measurement application,
if the measurement signal is received, draws skin moisture information corresponding to the received measurement signal based on the skin moisture measurement application, and
displays the skin moisture information.
